(19) United States
(12) Patent Application Publication
(10) Pub. No.: US 2021/0213082 A1
(43) Pub. Date: Jul. 15, 2021

(54) Botanical Synergistic Activated/Native and Equivalents Thereof

(71) Applicant: Fermented Farmer, LLC, Summertown, TN (US)

(72) Inventor: Jordan Seth Rubin, College Grove, TN (US)

(73) Assignee: Fermented Farmer, LLC, Summertown, TN (US)

(21) Appl. No.: 17/078,406

(22) Filed: Oct. 23, 2020

Related U.S. Application Data
(60) Provisional application No. 62/925,484, filed on Oct. 24, 2019.

(51) Int. Cl. A61K 36/185; A61K 31/192; A61K 31/05; A23L 33/105

(52) U.S. Cl. CPC A61K 36/185; A61K 31/192; A23V 2002/00; A23L 33/105; A61K 2236/37; A61K 31/05

(57) ABSTRACT

The invention is a nutraceutical composition which is admixed to contain two or more forms of botanicals selected from the group consisting of activated botanical, native botanical, aerial botanical and root botanical. For Cannabis/hemp forms according to the invention, the nutraceutical composition may contain one or more of native root Cannabis/hemp, activated root Cannabis/hemp, native “aerial” (above ground) portions of Cannabis/hemp and/or activated “aerial” portions of Cannabis/hemp.

### CROSS-REFERENCE TO RELATED APPLICATION

This patent application claims priority to, and incorporates herein by reference, U.S. Provisional Patent Application No. 62/925,484 filed 24 Oct. 2019.

### Field of the Invention

The invention pertains to particularly harvested and wielded Cannabis (hemp) plant parts for use in nutraceuticals and dietary supplements.

### BACKGROUND OF THE INVENTION

There is a long history as to many aspects of hemp's use as a medicinal botanical by various native cultures. Hemp (Cannabis sativa and C. indica) has been used in so many ways for so many millenia that the history and chemistry is extremely rich and varied. Even though hemp has had a celebrated history of use in traditional medical systems, there has been a void of hemp related nutraceutical innovation, as the plant has experienced a “dark” period for the better part of the last decade of unavailability to the average person. And, while hemp has experienced a renaissance in the later part of the 2010's, most of the development has focused on the hemp plant bud or flower as a source of beneficial medicinal compounds. However, there is a sound historical basis to use other parts of the hemp plant in the creation of medicinal, pharmaceutical and nutraceutical formulations. For example, a modicum of literature exists regarding Cannabis/Hemp roots, and the compounds and compositions therein and uses therefore. Cannabis/hemp roots do not contain Cannabidiol (CBD) or Tetrahydrocannabinol (THC) or their precursors, but instead contain valuable compounds including but not limited to terpenes and flavonoids. The terpenes include, without limitation, friedlein (a liver-protecting antioxidant) and epifriedelanol (an anti-tumor agent). Documentation of Cannabis/hemp root constituents and uses goes back at least to Ancient Greece if not earlier. In current U.S. practice, however, the root has largely if not completely fallen out of favor. It is not uncommon for Cannabis/hemp farmers to “turn over,” immediately after Cannabis harvesting, the unharvested roots in the soil, for composting in place. While this practice is great for soil management, a need remains for a current day appreciation of what Cannabis/hemp roots are capable of, biochemically and medicinally.

At the same time, the “aerial” parts of Cannabis or hemp, that is, the above ground plant structure as opposed to the root, has also been extensively documented throughout a long history not only for its constituents and uses, but also for techniques of extracting, activating, preserving, enhancing, or otherwise manipulating it to meet medical and market demand. A typical chemical reaction that occurs with Cannabis or hemp that contains tetrahydrocannibinolic acid (a carboxylic acid), as is widely known, is the decarboxylation to THC that occurs with various processing techniques, most notably heating. For this reason, in order to generate THC Cannabis/hemp has traditionally been heated or smoked, to realize the decarboxylation that “activates” the THCA to its active form, THC. A similar decarboxylation occurs, upon heating for example, to convert cannabidiolic acid (a carboxylic acid) to cannabidiol (CBD). In general, heating of these cannabinol compounds is understood to be an “activation” process, as opposed to the native forms which contain the carboxylic acid starting cannabinoids. Moreover, native forms are almost always or always the carboxylic acid forms, so “activation” of Cannabis or hemp is ubiquitous or at least extremely common within all the applications to which current-day Cannabis or hemp are utilized.

The above-described activation does not apply only to Cannabis/hemp. In recent years, research has been conducted that shows key bioactive constituents of certain spices, including but not limited to turmeric, peppers, ginger, and so forth, are variously subject to activation upon heating or other treatment, so that some compounds or compositions change to a more activated form upon treatment, whereas others disappear on heating. Suresh, D., “Effect of heat processing of spices . . . ,” Department of Biochemistry and Nutrition, Central Food Technological Research Institute, Mysore 570 020, India, Oct. 2, 2006 (published online at citeseerx.ist.psu.edu/viewdoc/download? doi=10.1.1.602.8886&rep=repl&type=pdf). So, even though heat generally activates the cannabinoids in hemp, for the purpose of this technology disclosure it should be borne in mind that for all botanicals, “activation” should be regarded as a partial chemical conversion, with some compounds or compositions achieving a more potentiated or bioactive form, while other compounds or compositions may be reduced in profile or may disappear altogether. Indeed, this “activation” phenonemon is a choice between Scylla and Charibdis in the sense that while activation causes beneficial conversion of some compounds and compositions in the plant, at the same time the same so-called activation inevitably deteriorates or obliterates other constituents that—statistically predictably—one did not want to lose or destroy. Therefore, a need remains for a protocol for botanical activation that maintains the best of both worlds—preserving the viability of the native compounds (such as terpene and flavonoids in Cannabis/hemp root, say) while at the same time realizing beneficial activation of compounds—not limited to cannabinoids—that convert to desired derivatives only after activation.

### SUMMARY OF THE INVENTION

In order to meet this heretofore unmet need, the present invention combines one or more of native and activated root parts of Cannabis/hemp with native and activated “aerial” parts of Cannabis/hemp, as described further below.

### DETAILED DESCRIPTION OF THE INVENTION

The present technology is a protocol for combining activated and native botanicals (or their equivalents) to achieve a “best of both” scenario, in which the resulting combination contains pro rata proportions of the native and activated chemical profiles. For example, a 50/50 mixture of native hemp root with heated/activated hemp root will preserve half of the original terpenes and flavonoids from the native hemp while containing fully half of the different constituents that occur only upon activation. The same ratio idea applies to “aerial” parts of Cannabis/hemp, that is, the plant parts above the ground (and, hence, in the air), with such activation yielding CBD and THC from CBDA and THCA, respectively. A 50/50 mixture of native hemp “aerial” parts with heated/activated hemp “aerial parts” will preserve half of the original terpenes and flavonoids from the native hemp while containing fully half of the different constituents that occur only upon activation but, with only “half activated” botanical, the remaining constituents from the native botanical are also present in rich, wider-spectrum array of nutritional or medicinal components. In one embodiment of the invention, the pro rata combination is four fold, especially in the instance of Cannabis/hemp. To create as broad a spectrum of constituents as possible, this technology combines a fraction of activated aerial hemp parts with a fraction of native aerial hemp parts, admixed with a fraction of activated root together with a fraction of native root. Typical ratios would be half and half activated versus native aerial Cannabis/hemp (or 10-50% activated and 90-50% native OR 10-50% native and 90-50% activated). The ratio of hemp “aerial” parts to hemp root is between 50/50 and 90/10, mimicking the naturally occurring ratio by weight of hemp “aerial” parts to hemp root and with a subsets of the hemp “aerial” parts and root being optionally activated and native as well. In an approximately four-fold combination of “aerial” parts and root, with each combined as fractionally both activated and native, not only is the broadest possible census of medicinal, pharmaceutical and nutraceutical constituents assembled all in one place, but the heretofore undiscovered co-factors present in the activated or native botanicals are maximally combined all together. As those in the field of pharmaceuticals and nutraceuticals already know, it is often not only a list of active constituents that is important, but bioavailability and synergy often depend on unknown/undiscovered co-factors and/or the admixture of desired nutrients and beneficial compounds with as many as possible of the compounds and compositions that are found with them in nature. In other words, present invention is able to overcome a lot of the myopia of ultimately counterproductive extraction, isolation and fractionization of a botanical—by making sure to preserve the fullest expression of the botanical's healing potential and ensuring the richest biochemical palette possible.

The above combinations are counter-intuitive (until one know them—everything is clear after one understands it) because in a pharmaceutical world where purity and derivatives are prized, yields of extracts and so forth are to be maximized, and potency is definitely “more is more” in our modern world, it is almost unthinkable deliberately to combine not only root but aerial hemp (or any other botanical) but to do so with fractions of either or both having been activated or preserved in native form In other words, in a world that values isolation fractionation, extraction and purification—adding back in large percentages of native botanicals with derivatized botanicals is, well, not where modern technology tends to go. And yet—by making these combinations to engineer constituent spectra—the best of both of activated and native compound mixtures is possible for the first time, unlike with any other botanical protocol in history.

While the invention is to be understood as described above, the following Example is illustrative of a portion of the present invention.

### Example 1

“HEMP 360 Oil,” a combination of equal parts native hemp root oil tincture with heated hemp root oil tincture, combined with aerial Cannabis/hemp plant parts or extracts containing 50% CBD and 50% CBDA, is made by mixing the constituents as described above. The desired CBD/CBDA ratio may be achieved by combining half native and half activated (heated) aerial Cannabis/hemp, or by selectively activating aerial hemp so as only to convert half the CBDA present to CBD, with the former being preferred, due to the greater preservation of native constituents to which no heat has been applied at all. Such an oil may be used as such or incorporated into other dosage forms including foods, beverages, dietary supplements and topical applications for humans and animals. Similarly, “HEMP 360 Capsules, Tablets or Powders” may include, for example, half native hemp and half activated hemp, so as to supply half CBD and half CBDA, together with all the activated and native compounds that result from either source. Ratios need not be 50:50 but can include ranges between 10-50 of one and 90-50 of the other. Activation can be by any means, including chemical, electromagnetic, heating, or any other energy input that creates activation of at least some of the native compounds or compositions in the botanical. Such a combination may include fractions of root, whether activated, native or both, ideally in ratios that resemble the preponderance of aerial parts to root, which is typically a 4:1 proportion by weight.

Although the invention has been described particularly, above, with specialized mention of ingredients and methods, the invention is only to be limited insofar as is set forth in the accompanying claims.

I claim:

1. A nutraceutical composition containing two or more forms of botanicals selected from the group consisting of activated botanical, native botanical, aerial botanical and root botanical.

2. The nutraceutical composition according to claim 1 wherein said active botanical and said native botanical are created simultaneously by partial activation of the same botanical material.

3. The nutraceutical composition according to claim 1 wherein said active botanical and said native botanical are created separately by activation of only one botanical.

4. The nutraceutical composition according to claim 1 wherein said botanical is hemp.

5. The nutraceutical composition according to claim 4 wherein all four of activated aerial hemp, native aerial hemp, native root hemp and activated root hemp are combined within the composition.

6. The nutraceutical composition according to claim 1, wherein said activated botanical is activated by heating prior to formulation in said composition.
